(19) United States
(12) Patent Application Publication
(10) Pub. No.: US 2021/0259952 A1
(43) Pub. Date: Aug. 26, 2021

(54) ORAL COMPOSITIONS AND MUCOADHESIVE THIN FILMS FORMED THEREFROM

(71) Applicant: BIOFILM LIMITED, Glasgow (GB)

(72) Inventors: Sarah Lindsay GRANT, Glasgow (GB); Mark Alexander LIVINGSTONE, Glasgow (GB); Dominic Gregory WALSH, Glasgow (GB); John Edward CULLEN, Glasgow (GB); Robert CRICHTON, Glasgow (GB); Melanie MACFARLANE, Glasgow (GB)

(21) Appl. No.: 17/254,900

(22) PCT Filed: Jun. 21, 2019
(86) PCT No.: PCT/GB2019/051766

§ 371 Date: Dec. 22, 2020

(30) Foreign Application Priority Data
Jun. 22, 2018 (GB) 1810244.2

(51) Int. Cl. A61K 9/00; A61K 47/32; A61K 47/36; A61K 47/02; A61K 45/06; A61K 31/05

(52) U.S. Cl. CPC A61K 9/006; A61K 47/32; A61K 31/05; A61K 47/02; A61K 45/06; A61K 47/36

(57) ABSTRACT

The present invention provides oral compositions. The compositions may be provided in a solid form, such as a film. The compositions can comprise one or more film forming polymers, one or more bioadhesive agents, one or more active agents, one or more polymeric solvents, and an aqueous solvent, with the proviso that the composition does not comprise benzocaine. The active agent(s) preferably are effective for a range of effects. The oral composition can be a solid or semi-solid composition up to a temperature of at least 40° C. Methods of providing such an oral compositions and methods of applying them to an oral or buccal cavity are also provided herein.

### FIELD OF THE INVENTION

The present invention relates to oral compositions useful for providing an occlusive barrier or for the delivery of active agents and more particularly to compositions suitable for formation of orally soluble, mucoadhesive films. Also provided is a method for the making the oral composition and a method of administering such an oral composition.

### BACKGROUND

Oral compositions are used for a variety of purposes, from the relief of pain by providing an occlusive barrier over a mucosal surface or administering an analgesic, to oral hygiene to keep the mouth clean and free from disease as well as to maintain and improve the aesthetic appearance of teeth. Such compositions may carry a variety of active agents, such as analgesics, anti-infectives, remineralisation agents for teeth, and dental bleaching agents to remove staining. This invention relates to a pliable and soluble mucoadhesive oral composition that is capable of acting as an occlusive barrier and/or providing targeted and long-lasting delivery of active agents to the buccal cavity or other oral mucosal surfaces and also to other oral surfaces such as the teeth and gums.

Current solutions for the topical treatment of a mucosal surface are mainly delivered in the form of liquids, pastes, gels, patches, disks and pressed tablets. These forms often dissolve, spread easily, or get dislocated and cause their effects throughout the oral mucosa. Other oral compositions may be provided in the solid state in the form of a strip or film, which adheres to teeth or mucosal surfaces upon contact with saliva. These solid strips are simple to use and may be applied by a practitioner or the individual desiring treatment. However, it can be difficult to incorporate an active agent into such strips or films. For instance, active agents may lack the requisite solubility to form a solid solution in the oral composition. Instead, the active agent precipitates as solid particulates in the composition, which may be unevenly distributed throughout the solid composition.

Accordingly, there is a desire and a need to provide an oral composition in which the active agent is evenly distributed. It is further desirable to provide an oral composition in which the active agent is present as a solid solution in the composition.

Furthermore, the existing compositions generally provide only a short-term active agent effect.

Accordingly, there is still a desire and a need to provide an oral composition that enables the active agent(s) to be delivered locally to the treatment site through the oral composition in a much more targeted fashion. It is further desirable to provide an oral composition that provides a long-lasting activity compared to existing products.

### SUMMARY OF THE INVENTION

The present disclosure relates to oral compositions that are either in a solid form or may be applied to form a substantially solid film inside the oral or buccal cavity. The solid composition is pliable, mucoadhesive, and slowly soluble within the oral or buccal cavity. The oral compositions overcome deficiencies of the prior art providing occlusion where required and enable the active agent(s) to be delivered locally to the treatment site through the mucoadhesive oral composition in a much more targeted fashion or systemically in the instance that the composition is delivered to the buccal mucosa. The composition can be a solid or semi-solid dosage form and preferably is in the form of a film that can be easily molded around a tooth or onto another surface, including substantially uneven surfaces, within the oral cavity to deliver an active agent thereto, with the proviso that the oral composition does not comprise benzocaine. It can provide good adhesion for maximum and targeted delivery of an active agent and hence, a more gradual coverage and longer duration of action.

In one or more embodiments, the present disclosure particularly provides an oral composition comprising: one or more film forming polymers in a total amount of about 40% to about 80% by weight; one or more bioadhesive agents in a total amount of about 0.5% to about 10% by weight; one or more active agents in a total amount of about 0.01% to about 35% by weight; one or more polymeric solvents in which the one or more active agents is solubilized, the one or more polymeric solvents being in a total amount of about 0.1% to about 30% by weight; and an aqueous medium in an amount of about 0.5% to about 15% by weight; each of the foregoing amounts being based on the total weight of the oral composition, with the proviso that the oral composition does not comprise benzocaine. The oral composition preferably is in the form of a film having a thickness of about 50 μm to about 500 μm. In further embodiments, the oral composition may be defined in relation to any one or more of the following statements, which can be combined in any order and number.

The one or more film forming polymers can comprise one or more of a polyvinylpyrrolidone and a polysaccharide. The polysaccharide may be one or more of the group comprising pullulan, pectin, starch, alginic acid or a derivative thereof and cellulose derivative.

The one or more film forming polymers can be selected from the group consisting of a polyvinylpyrrolidone, pullulan, pectin, starch, carboxyalkyl cellulose or a salt thereof, hydroxyalkyl cellulose or a salt thereof, in which the alkyl group is independently selected from C₁₋₅ alkyl, alginic acid, salt of alginic acid, and combinations thereof.

The one or more film forming polymers can include a polyvinylpyrrolidone in an amount of about 20% to about 40% by weight and pullulan in an amount of about 20% to about 40% by weight, based on the total weight of the oral composition.

The one or more bioadhesive agents can comprise one or more of a polyacrylic acid and derivatives thereof and a gum.

The polyacrylic acid and derivatives thereof may be one or both of a polycarbophil and a carbomer. The gum may be a natural gum or a synthetic gum.

When the one or more bioadhesive agents include a polycarbophil it can be present in an amount of about 0.25% to about 5% by weight, based on the total weight of the oral composition.

The composition further can comprise one or more opacifiers in a total amount of about 0.02% to about 2% by weight based on the total weight of the composition.

The composition may further comprise a physiologically acceptable plasticizer. In one embodiment, the plasticizer is a hydrophilic plasticizer, such as one or more of the group comprising a polyol such as glycerol, monosaccharides, oligosaccharides, lipids, sorbitol and sorbitan. The preferred plasticizer is glycerol. Preferably the plasticizer is present in a proportion of from about 0.25% to about 15% by weight of the oral composition. For instance, the composition may further comprise glycerol in an amount of about 0.25% to about 5% by weight, based on the total weight of the oral composition.

The one or more active agents may be selected from the group comprising a pharmaceutical, a bleaching agent, an anti-staining agent, a dental remineralization agent, a dental desensitizing agent, an anti-caries agent, an anti-malodour agent, an essential oil, a herbal remedy, a botanical extract of cannabinoids and an anti-calculus agent, with the proviso that the active agents does not comprise benzocaine. In some embodiments, the one or more active agents may be selected from the group comprising a pharmaceutical, a bleaching agent, an anti-staining agent, a dental remineralization agent, a dental desensitizing agent, an anti-caries agent, an anti-malodour agent and an anti-calculus agent, with the proviso that the active agents does not comprise benzocaine.

The pharmaceutical may be one or more of the group comprising pain relievers, anti-inflammatory agents, sedatives, hypnotics, antibiotics, antidiabetics, antihypertensives, anti-osteoporosis agents, antithrombotic agents, anti-infective agents such as antivirals, antibacterials and antifungals, anticholinergic agents, anxiolytic agents, adrenergics, antipsychotics, anti-parkinsonism agents, anticonvulsants, antiepileptics, CNS stimulants, antianginal agents, antiarrhythmics, antihyperlipidemic drugs, diuretics, antiasthmatics, anticoagulants, antianemia agents, vitamins, hormones including natural hormones and synthetic hormones, antihistaminics, anticancer agents, antiallergics, antiarthritis agents, antialzheimers' agents, vasopressin antagonists, anticonvulsants, steroids, anesthetics, thrombolytics, antacids, proton pump inhibitors, protease inhibitors, platelet aggregation inhibitors, mucolytics, antimalarials, antiemetics, laxatives, expectorants, enzymes, contraceptives, bronchodilators, antitussives, antimigraine agents, anthelmintics, biomolecules, salivating agents, anti-foaming agents, anti-snoring agents and anorexiants, with the proviso that the pharmaceutical does not comprise benzocaine.

The one or more active agents can be included in an amount of about 0.01% to about 35% by weight based on the total weight of the oral composition.

The one or more polymeric solvents can comprise at least two polyalkylene glycols, wherein the alkylene group of each polyalkylene glycol is independently selected from C₂-C₅ alkylene. Preferably the alkylene group is selected from ethyl or propyl.

The at least two polyalkylene glycols may comprise a first and a second polyalkylene glycol wherein the first and second polyalkylene glycols are not the same i.e. they differ in one or both of their molecular weight and the chain length of alkylene group. Thus, the at least two polyalkylene glycols may comprise two polyalkylene glycols having the same alkylene group but different molecular weights or may comprise two polyalkylene glycols having different alkylene groups.

The at least two polyalkylene glycols may comprise a lower molecular weight polyalkylene glycol and a higher molecular weight polyalkylene glycol.

The lower molecular weight polyalkylene glycol may have a molecular weight in the range of from 200 to ≤1,500 gram per mole and the higher molecular weight polyalkylene glycol may have a molecular weight in the range of >1,500 to 20,000 grams per mole. Typically, the lower molecular weight polyalkylene glycol may have a molecular weight in the range of from 400 to 1,000 grams per mole and the higher molecular weight polyalkylene glycol may have a molecular weight in the range of from 1,600 to 10,000 grams per mole.

The first polyalkylene glycol, which may be the lower molecular weight polyalkylene glycol, can be present in an amount of about 1% to about 20% by weight, and the second polyalkylene glycol, which may be the higher molecular weight polyalkylene glycol can be present in an amount of about 1% to about 20% by weight, based on the total weight of the oral composition.

The one or more polymeric solvents can comprise polyethylene glycol (PEG).

The one or more polymeric solvents can include a first polyethylene glycol (PEG) having a molecular weight of 1,500 grams per mole or less and a second polyethylene glycol (PEG) having a molecular weight of 2,000 grams per mole or greater.

The first PEG can be present in an amount of about 1% to about 20% by weight, and the second PEG can be present in an amount of about 1% to about 20% by weight, based on the total weight of the oral composition.

The aqueous medium may comprise water. The aqueous medium may consist essentially of water. The aqueous medium may consist of water.

The aqueous medium can be present in an amount of about 5% to about 12% by weight, based on the total weight of the oral composition.

The composition further can comprise one or more opacifiers in a total amount of about 0.02% to about 2% by weight based on the total weight of the composition.

The opacifier can include titanium dioxide.

The film can be configured to dissolve substantially completely in contact with oral or buccal mucosa in a time of about 15 minutes to about 150 minutes, typically from about 15 minutes to about 120 minutes.

In one or more embodiments, the present disclosure further can relate to a method of making an oral composition. Such method can comprise: solubilizing one or more active agents in one or more polymeric solvents while heating to a temperature of about 50° C. to about 120° C. to form a premix; mixing into the premix each of the following components to form a liquid composition: one or more film forming polymers; one or more bioadhesive agents; and water; coating the liquid composition onto a backing sheet to form a layer of the composition having a thickness of about 100 μm to about 3,000 μm; and drying the layer to form a film of the oral composition having a total water content of about 5% to about 15% by weight based on the total weight of the film, with the proviso that the composition does not comprise benzocaine.

In further embodiments, the method of preparing the oral composition may be defined in relation to any one or more of the following statements, which can be combined in any order and number.

The film of the oral composition can comprise: a total of about 0.01% to about 35% by weight of the one or more active agents; a total of about 0.1% to about 30% by weight of the one or more polymeric solvents: a total of about 40% to about 80% by weight of the one or more film forming polymers; and a total of about 0.5% to about 10% by weight of the one or more bioadhesive agents; each of the foregoing amounts being based on the total weight of the film of the oral composition.

The drying can comprise applying heated air to the layer of the composition on the backing sheet for a time of about 20 minutes to about 150 minutes, typically about 20 to about 120 minutes.

The heated air may be applied from one or both of above and below the layer of the composition. In one embodiment the heated air can be applied only from above the layer of the composition.

The drying can be carried out by passing the layer of the composition on the backing sheet through a drying tunnel.

The drying may form a layer of composition having a thickness of about 50 μm to about 500 μm.

In some embodiments, drying can be carried out to achieve a composition with an overall water or moisture content of about 1% to about 15%, about 3% to about 15%, or about 5% to about 15%, or about 5% to about 12% by weight, based on the total weight of the overall oral care composition.

The method further can comprise cutting the film of the oral composition into individual strips having a width of about 5 mm to about 25 mm and having a length of about 10 mm to about 85 mm, typically of about 15 to about 60 mm. Preferably the film is cut to a length of about 60 mm.

In one or more embodiments, the present disclosure further can relate to an oral composition obtainable by the method of making and its embodiments.

In one or more embodiments, the present disclosure further can relate to a method of administering an active agent to an oral or buccal cavity comprising at least the step of:

applying the oral composition as described herein to a portion of an oral or buccal cavity.

In further embodiments, the method of administering the active agent may be defined in relation to any one or more of the following statements, which can be combined in any order and number.

The one or more active agents may be selected from the group comprising a pharmaceutical, a bleaching agent, an anti-staining agent, a dental remineralization agent, a dental desensitizing agent, an anti-caries agent, an anti-malodour agent and an anti-calculus. The one or more active agents may be selected from the group comprising a bleaching agent, an anti-staining agent, a dental remineralization agent, a dental desensitizing agent, an anti-caries agent, an anti-malodour agent and an anti-calculus.

The method may be a cosmetic method, such as a solely cosmetic method. For instance, the one or more active agents may be one or both of a bleaching agent and an anti-staining agent.

The method may be a method of treatment. For instance, the one or more active agents may be a pharmaceutical.

### DETAILED DESCRIPTION OF THE INVENTION

The present disclosure now will be described more fully hereinafter. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise. As used in this specification, the term “comprising” may be replaced by the term “consisting essentially of” or “consisting of”.

As used in this specification, the term “molecular weight” refers to weight average molecular weight.

The present invention is directed to compositions that are configured for formation of thin films. The formed thin films are mucoadhesive, pliable, orally soluble, and adapted for delivery of one or more active agents to the buccal cavity or an oral surface over a prolonged period of time. In particular, the thin films are suitable for providing delivery of an active agent such as an analgesic or the like that is adapted for providing treatment to the buccal cavity or oral surface, such as treatment of a medical indication, for instance to provide temporary pain relief in an area of the buccal or the oral cavity, such as the tooth and/or gums in relation to a toothache and/or to an area of the mouth in relation to a mouth sore. The treatment may also relate to a cosmetic method, for instance to provide tooth whitening. Thus, treatment of an oral or buccal cavity includes the treatment of teeth.

The thin film can be sized to be able to cover a suitably large area of the oral surface in order to provide the localized delivery of the active agent without substantial migration of the active agent to other portions of the oral cavity. Thus, the thin films provide targeted delivery of the active agent to achieve a more gradual coverage and longer duration of action to the intended purpose—e.g., pain relief. Alternatively, where buccal delivery of the active agent is desirable, the films can provide sustained delivery of the active agent to the buccal mucosa.

In one or more embodiments, the present disclosure can provide oral compositions. In preferred embodiments, the oral compositions are provided in substantially solid forms with a substantially uniform shape, such as pastilles, sheets, and films. The oral compositions preferably can be a solid or semi-solid composition up to a temperature of at least 40° C. Pastilles, sheets, and films can each be similarly configured as having a length and a width that is substantially greater than a thickness thereof. For example, pastilles can be thicker than sheets while sheets can be thicker than films. In any case, it is preferred that the oral composition can be applied to an area of the oral cavity so that the oral composition will be in the form of a substantial solid after application to the area of the oral cavity. Preferably, the oral composition is provided in a substantially solid form before application to the area of the oral cavity to provide for simplified application by the user. The oral composition, being in a substantially solid form after application to the area of the oral cavity, can be configured to dissolve over time to release an active agent locally at the site of application in the oral or buccal cavity.

Dissolution time can vary as otherwise described herein. In particularly preferred embodiments, the oral composition is provided in the form of a film having a thickness of about 50 μm to about 500 μm, about 100 μm to about 500 μm, about 120 μm to about 350 μm, or about 140 μm to about 250 μm. In some embodiments, the film can have a thickness of about 500 μm or less, about 400 μm or less, about 300 μm or less, or about 200 μm or less. In such cases, it is understood that the film has a minimum thickness that is greater than or equal to 50 μm. The film can be provided in the form of strips having a width and length that provide for ease of application to the area of the oral cavity, ease of handling generally, and sufficiently broad area of coverage in the oral cavity to provide sufficient local delivery of the active agent. For example, the film can have a width of about 5 mm to about 25 mm, about 10 mm to about 20 mm, or about 12 mm to about 18 mm. The film can have a length of about 10 mm to about 85 mm, about 15 mm to about 60 mm, or about 20 mm to about 30 mm. Similar sizing may be applied to other solid forms, such as pastilles and sheets. It is understood, however, that when the oral composition is in forms having a greater thickness, the width and/or length of the oral composition may be lesser than when in the form of a film in order to deliver a substantially equal amount of the active agent to the local area of the oral cavity.

In one or more embodiments, an oral composition according to the present disclosure specifically can comprise one or more film forming polymers. The film forming polymer(s) can be specifically configured to cause the oral composition to take on the desired solid form when dried to a sufficiently low water content. For example, the desired solid form can be a film in preferred embodiments as defined above, or may be a sheet or pastille form having a thickness that is greater than the thickness of a film. Suitable film forming polymers include one or more of a polyvinylpyrrolidone and a polysaccharide. Suitable polysaccharides include pullulan, pectin, starch, alginic acid or a derivative thereof and a cellulose derivative. Suitable alginic acid derivatives includes salts of alginic acid, such as alginates. Suitable cellulose derivatives include carboxyalkyl cellulose or a salt thereof and hydroxyalkyl cellulose or a salt thereof, in which the alkyl group of the carboxyalkyl cellulose or the hydroxyalkyl cellulose is independently selected from C₁₋₅ alkyl, preferably methyl, ethyl or propyl. Other known film forming polymers may be used in combination with or as a replacement for any of the foregoing. In preferred embodiments, however, it has been found that particularly suitable films can be prepared when the oral composition comprises one or more polymers selected from the group consisting of a polyvinylpyrrolidone, pullulan, hydroxypropyl cellulose, pectin, and combinations thereof. In some embodiments, only a single film forming polymer may be utilized. In further embodiments, it can be useful to use a combination of two film forming polymers, three film forming polymers, or even more film forming polymers. Preferably, the film forming polymer can include at least pullulan, at least a polyvinylpyrrolidone, or at least both of pullulan and a polyvinylpyrrolidone.

The film forming polymer(s) can be present in a total amount of about 40% to about 80% by weight based on the total weight of the oral composition. The total amount may be accounted for by a single film forming polymer or may be the combination of two or more film forming polymers. Preferably, the film forming polymer(s) are present in a total amount of about 45% to about 75% by weight, about 50% to about 70% by weight, or about 55% to about 65% by weight, based on the total weight of the oral composition. In some embodiments, two film forming polymers can be used, each of which is independently present in an amount of about 20% to about 40% or about 25% to about 35% by weight based on the total weight of the oral composition. For example, the oral composition can comprise a polyvinylpyrrolidone in an amount of about 20% to about 40% or about 25% to about 35% by weight and also comprise pullulan in an amount of about 20% to about 40% or about 25% to about 35% by weight, based on the total weight of the oral composition.

In some embodiments, two film forming polymers may be utilized in a defined ratio. For example, a polyvinylpyrrolidone and a polysaccharide may be combined in a ratio of 3:1 to 1:3, 2:1 to 1:2, or about 1:1. Like ratios may be utilized for any combination of two film forming polymers as described herein. Such ranges were found to provide optimal adhesion over time whilst also providing sufficient loading of the active agent in the composition.

In one or more embodiments, the oral composition can comprise one or more bioadhesive agents. The bioadhesive agent(s) can be any material that is adapted to cause the oral composition, particularly when in a solid form factor, to adhere to oral tissue, particularly oral mucosa, such as the gums. The one or more bioadhesive agents can comprise one or more of a polyacrylic acid and derivatives thereof and a gum. The gum may be a natural gum or a synthetic gum. The natural gum may be selected from carrageenan, tragacanth and polysaccharide gums. More preferably, when the one or more bioadhesive agents comprise a natural gum, it is tragacanth gum. The polyacrylic acid may be a high molecular weight polyacrylic acid such as a carbomer. Polymers of acrylic acid may be either crosslinked or uncrosslinked. Examples of crosslinking agents include allyl ether pentaerythritol, allyl ethers of sucrose or ally ethers of propylene. Crosslinked polymers of acrylic acid are sold under the trade name Carbopol® by Lubrizol Corporation. The polyacrylic acid derivative may be a polyacrylic acid provided as a salt, such as an ammonium, alkali metal or alkaline earth metal salt. Cross-linked polyacrylic acid, such as those cross-linked with divinyl glycol may be provided as salts such as alkaline earth metal salts, particularly calcium salts, also known as polycarbophils. Thus, the polyacrylic acid and derivatives thereof may be one or both of polycarbophil and carbomer.

Other components of the composition may also exhibit bioadhesive properties. Thus, one or more components used in the present compositions for other purposes as defined herein may also provide a secondary, bioadhesive effect. For example, one or more film forming polymers (e.g., polyvinylpyrrolidones) and/or one or more polymeric solvents (e.g., PEG) may provide both the primary function as defined herein as well as providing a secondary function of bioadhesion. It is thus understood that use herein of the term “bioadhesive agent” relates to a material that has a primary function in the overall composition of providing bioadhesion. As noted, however, other components of the composition may have a secondary function of providing bioadhesion as well. However, for the purposes of the amounts of the bioadhesive agent present in the composition, these may refer to bioadhesive agents which are performing their primary function and do not include those which enhance bioadhesion as a secondary function. Thus, if an agent is disclosed herein as performing a different function, it is not considered as a bioadhesive agent when calculating the amount of the bioadhesive agent.

In some embodiments, only a single component having the primary function of a bioadhesive agent may be utilized. In further embodiments, it can be useful to use a combination of two or more components having the primary function of a bioadhesive agent. Still further, the compositions may include a single component having the primary function of a bioadhesive agent in combination with one or more components having a secondary function of bioadhesion. Likewise, the compositions may include two or more components having the primary function of a bioadhesive agent in combination with one or more components having a secondary function of bioadhesion. Preferably, the present compositions can include at least a polycarbophil as a bioadhesive agent.

The bioadhesive agent(s) can be present in a total amount of about 0.5% to about 10% by weight based on the total weight of the oral composition. The total amount may be accounted for by a single bioadhesive agent or may be the combination of two or more bioadhesive agents. Preferably, the bioadhesive agent(s) are present in a total amount of about 0.75% to about 9% by weight, about 1% to about 8% by weight, or about 3% to about 7% by weight, based on the total weight of the oral composition. In some embodiments, two bioadhesive agents can be used, each of which is independently present in an amount of about 0.25% to about 5% or about 1% to about 4% by weight based on the total weight of the oral composition. For example, the oral composition can comprise a polycarbophil in an amount of about 0.25% to about 5% or about 1% to about 4% by weight and also comprise a polyol in an amount of about 0.25% to about 5% or about 1% to about 4% by weight, based on the total weight of the oral composition. It is understood that the foregoing amounts can expressly exclude the contents of other components otherwise described herein that only provide a secondary function of bioadhesion.

In some embodiments, a bioadhesive agent and a plasticizer may be utilized in a defined ratio. For example, a polycarbophil and a polyol may be combined in a ratio of 3:1 to 1:3, 2:1 to 1:2, or about 1:1. Like ratios may be utilized for any combination of bioadhesive agent and plasticizer.

In various embodiments, one or more active agents can be included in the oral composition. The one or more active agents can be configured to provide any number of desired effects. In certain embodiments, the active agent(s) can be configured to provide pain relief and/or soothing and/or cooling effects. The one or more active agents can be present in an amount suitable for providing the desired effect.

The active agent may be selected from the group comprising a pharmaceutical, a bleaching agent, an anti-staining agent, a dental remineralization agent, a dental desensitizing agent, an anti-caries agent, an anti-malodour agent or an anti-calculus agent, with the proviso that the active agent does not comprise benzocaine. Thus, the active agent may be a pharmaceutical, or a cosmetic active agent, such as a solely cosmetic active agent.

The one or more pharmaceutical agents may be one or more of pain relievers, anti-inflammatory agents, sedatives, hypnotics, antibiotics, antidiabetics, antihypertensives, anti-osteoporosis agents, antithrombotic agents, anti-infective agents such as antivirals, antibacterials and antifungals, anticholinergic agents, anxiolytic agents, adrenergics, antipsychotics, anti-parkinsonism agents, anticonvulsants, antiepileptics, CNS stimulants, antianginal agents, antiarrhythmics, antihyperlipidemic drugs, diuretics, antiasthmatics, anticoagulants, antianemia agents, vitamins, hormones including natural hormones and synthetic hormones, antihistaminics, anticancer agents, antiallergics, antiarthritis agents, antialzheimers' agents, vasopressin antagonists, anticonvulsants, steroids, anesthetics, thrombolytics, antacids, proton pump inhibitors, protease inhibitors, platelet aggregation inhibitors, mucolytics, antimalarials, antiemetics, laxatives, expectorants, enzymes, contraceptives, bronchodilators, antitussives, antimigraine agents, anthelmintics, biomolecules, salivating agents, anti-foaming agents and anti-snoring agents and anorexiants, with the proviso that the one or more pharmaceutical agents are not benzocaine.

Examples of pain relievers include non-steroidal anti-inflammatory drugs, acetaminophens, cannabinoids, combined sleep aids and pain reliever blends, opioids, muscle relaxants, benzodiazepines, nonbenzodiazepine hypnotics, anticonvulsants and antidepressants, with the proviso that the pain reliever is not benzocaine. Examples of non-steroidal anti-inflammatory drugs include aspirin, ibuprofen, ketoprofen and naproxen. Examples of acetaminophens include tylenol and panadol. Examples of cannabinoids include tetrahydrocannabinol, cannabidiol and cannabinol. Examples of combined sleep aids and pain reliever blends include ibuprofen with diphenhydramine, and acetaminophen with diphenhydramine. Examples of opioids include codeine, morphine, oxycodone, oxycodone with acetaminophen and hydrocodone with acetaminophen. Examples of muscle relaxants include cyclobenzaprine, baclofen, metaloxalone and carisoprodol. Examples of benzodiazepines include lorazepam, flurazepam, triazolom, clonazepam, temazepam and diazepam. Examples of nonbenzodiazepine hypnotics include zolpidem, eszopiclone and zaleplon. Examples of anticonvulsants include tiagabine, carbamazepine, gabapentin and topiramate. Examples of antidepressants include nortriptyline, trazodone, amitriptyline, nefazodone and duloxetine. Exemplary biomolecules include bacteriocins, antibodies and enzymes.

The anti-infective agent may be selected from one or more of the group comprising an antibacterial, an antiviral and an antifungal. The anti-infective preferably comprises an antibacterial, and particularly an antibacterial for the treatment of biofilms. Antibacterials may be one or more compounds selected from the group comprising benzoyl peroxide, triclosan, chlorhexidine, copper-, zinc- and stannous salts such as zinc citrate, sodium zinc citrate and stannous pyrophosphate, sanguinarine extract, metronidazole, quaternary ammonium compounds and salts thereof. Quaternary ammonium compounds suitable as antibacterials include cetylpyridinium chloride; bis-guanides such as chlorhexidine digluconate, hexetidine, octenidine, alexidine; and halogenated bisphenolic compounds such as 2,2′methylenebis-(4-chloro-6-bromophenol). A preferred antibacterial is chlorohexidine or a salt thereof, such as chlorohexidine dihydrochloride, chlorohexidine diacetate or chlorohexidine digluconate.

Anti-inflammatory agents may be steroidal anti-inflammatory agents or non-steroidal anti-inflammatory agents. Preferably, the anti-inflammatory agents comprise one or more compounds selected from the group comprising ibuprofen, flurbiprofen, aspirin and indomethacin.

The one or more active pharmaceutical agents may be one or more of amlodipine, diazepam, paracetamol, aspirin, ciprofloxacin, dicyclomine, celecoxib, alendronate, caffeine including encapsulated caffeine, diacerein, acyclovir, fluconazole, epinephrine, divalproex, methylphenidate, flecainide, metoprolol, fenofibrate, hydrochlorothiazide, montelukast, heparin, warfarin, hemoglobin, iron, ascorbic acid, leutinizing hormone, bicalutamide, donepezil, tolvaptan, cortisones, lidocaine, calcium carbonate, saquinavir, bromhexine, promethazine, bisacodyl, pancreatin, ethinyl estradiol, salbutamol, diphenhydramine, sumatriptan, diclofenac, metronidazole, orlistat, ibuprofen, indomethacin, ketorolac, tramadolol, oxcarbazepine, pioglitazone, rosiglitazone, miglitol, vildagliptin, sitagliptin, repaglinide, voglibose, alprazolam, chlorpromazine, cimetidine, pseudoephedrine, naproxen, piroxicam, atenolol, benazepril, captopril, lisinopril, fosinopril, enalapril, furosemide, indapamide, atenolol, felodipine, verapamil, cartenolol, carvedilol, cerivastatin, diltiazem, fluvastatin, irbesartan, candesartan, methyldopa, nicotine, reserpine, bupropion, fluoxetine, paroxetine, escitalopram, sertraline, amitryptiline, imipramine, fexofenadine, clopidogrel, entacapone, levodopa, carbidopa, levetiracetam, venlafaxine, duloxetine, lisinopril, losartan, lovastatin, niacin, menthol, pravastatin, ramipril, simvastatin, atorvastatin, valsartan, telmisartan, sildenafil, tadalafil, vardenafil, esomeprazole, famotidine, omeprazole, pantoprazole, rabeprazole, ranitidine, simethicone, artesunate, amodiaquine, benazepril, misoprostol, metformin, glipizide, tetrahydrocannabinol, cannabidiol, cannabinol and their pharmaceutically acceptable salts.

The bleaching agent bleaching agent may be an organic or inorganic bleaching agent. Examples of organic bleaching agents are organic peroxy acids including 6-(phthalimido)peroxyhexanoic acid and benzoyl peroxide. Organic peroxy acids may be provided with a stabilizing agent such as dipicolinic acid or sodium stannate. Examples of inorganic bleaching agents include hydrogen peroxide and peroxymonosulphuric acid and chlorites. Preferably the chlorite is an alkali metal chlorite, such as sodium chlorite. Preferably, the peroxide is selected from one or more of the group comprising hydrogen peroxide, peracids, such as percarboxylic acids, benzoyl peroxide, carbamide peroxide, sodium perborate and sodium percarbonate. Most preferably the bleaching agent comprises hydrogen peroxide. Preferably, when the bleaching agent comprises hydrogen peroxide, at least a portion of the hydrogen peroxide is present with a polyvinyl pyrrolidone as a complex. The bleaching agent may be present entirely complexed with a polyvinyl pyrrolidone film forming agent, or a portion of the bleaching agent may be present with the polyvinyl pyrrolidone in a complex with the remainder of the bleaching agent is present in the composition in uncomplexed form i.e. unbound to polyvinyl pyrrolidone. The hydrogen peroxide can form hydrogen bonds with the carbonyl group of the pyrrolidone ring to form the complex. One hydrogen peroxide molecule may form a hydrogen bond with at least one carboxyl group of the pyrrolidone. However, one hydrogen peroxide molecule may also form two hydrogen bonds, each one with the carbonyl group of two adjacent pyrrolidone rings. Thus, the molar ratio of pyrrolidone groups to hydrogen peroxide in such a complex may be in the range of about 1:1 to 2:1. This results in complexes which may comprise from 15 to 20 wt. % by weight hydrogen peroxide and from 80 to 85 wt. % by weight polyvinyl pyrrolidone depending upon the degree of coordination between hydrogen peroxide and carbonyl groups. The polyvinyl pyrrolidone polymer in the complex may be one or more selected from the group comprising uncrosslinked polyvinyl pyrrolidone, crosslinked polyvinyl pyrrolidone, a copolymer of vinyl pyrrolidone and acrylic acid, a copolymer of vinyl pyrrolidone and vinyl acrylate, or alkylated polyvinyl pyrrolidone. The polyvinyl pyrrolidone may preferably have a weight average molecular weight in the range of from 1 million to 1.5 million, still more preferably about 1.3 million. Suitable pyrrolidone polymer complexes with hydrogen peroxide are sold under the trade name Peroxydone™ by Ashland.

The anti-staining agent may be a polyphosphate, such as sodium hexametaphosphate, sodium tripolyphosphate, or a combination thereof.

The dental remineralisation agent may be a bioactive glass. Bioactive glasses can facilitate the remineralisation to tooth enamel. Consequently, they may also function as tooth desensitizing agents. Preferably the bioactive glass is a phosphosilicate, such as calcium sodium phosphosilicate. More preferably the bioactive glass is a milk protein comprising casein phosphopeptide amorphous calcium phosphate, such as Recaldent™ of Cadbury Enterprises Pte Ltd.

The dental desensitizing agent may be selected from one or more of the group comprising an oxalate such as an alkali metal oxalate, arginine, alkali metal citrate, alkali metal chloride, alkali metal tartrate, alkali metal bicarbonate, alkali metal nitrate, salts of strontium and a fluoride such as an alkali metal fluoride. Preferably the alkali metal is sodium or potassium. More preferably the desensitizing agent is selected from one or more of the group comprising potassium citrate, potassium chloride, potassium tartrate, potassium bicarbonate, potassium oxalate, potassium nitrate and sodium fluoride.

Anti-caries agents may be one or more compounds selected from the group comprising sodium fluoride, stannous fluoride, amine fluorides, monosodium fluorophosphate, casein and plaque buffers. Exemplary plaque buffers include urea, calcium lactate, calcium glycerophosphate and strontium polyacrylates.

Anti-malodour agents may be one or more compounds selected from the group comprising chlorhexidine, amine fluorides, stannous salts, metal ions with a high affinity for sulphur e.g. zinc, blends of aromatic ethers or alcohols with essential oils, and essential oils. Products such as Meridol, CB12 are blends of malodour actives.

Anti-calculus agents may be one or more compounds selected from the group comprising alkali metal pyrophosphates, hypophosphite containing polymers, organic phosphonates and phosphocitrates.

A botanical extract of cannabinoids may be cannabidiol extract or resin from cannabis, such as one or more compounds extracted and isolated from cannabis. The botanical extract may include one or more compounds selected from the group comprising terpene, tetrahydrocannabinols (THCs), cannabidiol (CBD), and/or cannabinoids.

The active agent(s) can be present in a total amount of about 0.01% to about 35% by weight based on the total weight of the oral composition. The total amount may be accounted for by a single active agent or may be the combination of two or more active agents. Preferably, the active agent(s) are present in a total amount of about 0.1% to about 30% by weight, about 1% to about 25% by weight, or about 5% to about 20% by weight, based on the total weight of the oral composition. In some embodiments, two active agents can be used, each of which is independently present in an amount of about 1% to about 15% or about 1% to about 10% by weight based on the total weight of the oral composition.

While various polymeric materials can be particularly suitable for use herein as film-forming polymers, one or more active agent(s) useful herein may not be particularly soluble in the film-forming polymer. Accordingly, in various embodiments, the oral compositions of the present disclosure further can include one or more polymeric solvents in which the one or more active agents is solubilized. The polymeric solvents can include any polymeric material in which the active agent exhibits higher solubility when compared to the film-forming polymer and which is suitable for intermixing with the film-forming polymer.

The one or more polymeric solvents can comprise at least two polyalkylene glycols, wherein the alkylene group of each polyalkylene glycol is independently selected from C₂-C₅ alkylene i.e. ethylene, propylene, butylene, pentylene and the structural isomers thereof, such as n-propylene, i-propylene etc. Preferably the alkylene group is selected from ethyl or propyl. The at least two polyalkylene glycols may comprise a first and a second polyalkylene glycol wherein the first and second polyalkylene glycols are not the same i.e. they differ in one or both of their molecular weight and the chain length of alkylene group. Thus, the at least two polyalkylene glycols may comprise two polyalkylene glycols having the same alkylene group but different molecular weights or may comprise two polyalkylene glycols having different alkylene groups and the same or different molecular weights. The at least two polyalkylene glycols may comprise a lower molecular weight polyalkylene glycol and a higher molecular weight polyalkylene glycol. For the avoidance of doubt the terms “lower” and “higher” are relative to one another, such that the lower molecular weight polyalkylene glycol has a molecular weight which is less than that of the higher molecular weight polyalkylene glycol. The lower molecular weight polyalkylene glycol may have a molecular weight in the range of from 200 to ≤1,500 gram per mole and the higher molecular weight polyalkylene glycol may have a molecular weight in the range of >1,500 to 20,000 grams per mole. Typically, the lower molecular weight polyalkylene glycol may have a molecular weight in the range of from 400 to 1,000 grams per mole and the higher molecular weight polyalkylene glycol may have a molecular weight in the range of from 1,600 to 10,000 grams per mole.

At least two polyalkylene glycols of different molecular weights, particularly those in the ranges discussed above, provide an advantageous combination of properties including solubilization of the active agent and stabilization, adhesion and plasticization of the film. The lower molecular weight polyalkylene glycol enhances the solubility of the active agent in the composition. The higher molecular weight polyalkylene stabilizes the solid solution of the active agent in the composition.

In certain embodiments, it can be particularly useful for the polymeric solvent to include one or more grades of polyethylene glycol (PEG). Different grades of PEG can be characterized in relation to the average molecular weight thereof (e.g., such as based on a weight average molecular weight). It can be particularly beneficial to utilize two different grades of PEG as the polymeric solvent. As such, the polymeric solvent can comprise a first PEG having a first average molecular weight and a second PEG having a second average molecular weight. The first average molecular weight is different from the second average molecular weight. Preferably, the second average molecular weight is greater than the first average molecular weight by a factor of at least 1.5, a factor of at least 2, a factor of at least 4, a factor of at least 5, a factor of at least 8, or a factor of at least 10.

In some embodiments, first PEG useful as a polymeric solvent can be characterized in terms of having a lower molecular weight relative to the second PEG. As an example, the first PEG may have an average molecular weight of about 1,500 grams per mole or less, about 1,000 grams per mole or less, or about 800 grams per mole or less. Preferably, in such embodiments, the first PEG has an average molecular weight of at least 200 grams per mole. In certain embodiments, the first PEG may have an average molecular weight of about 200 grams per mole to about 1,000 grams per mole, about 200 grams per mole to about 800 grams per mole, or about 250 grams per mole to about 600 grams per mole. In other embodiments, the first PEG may have an average molecular weight of about 200 grams per mole to about 1,500 grams per mole, about 250 grams per mole to about 1,500 grams per mole, or about 300 grams per mole to about 1,200 grams per mole.

In some embodiments, a second PEG useful as a polymeric solvent can be characterized in terms of having a higher molecular weight relative to the first PEG. As an example, the second PEG may have an average molecular weight of about 2,000 grams per mole or more, about 2,500 grams per mole or more, or about 3,000 grams per mole or more. Preferably, in such embodiments, the second PEG has an average molecular weight of no more than about 20,000 grams per mole. In certain embodiments, the second PEG may have an average molecular weight of about 2,000 grams per mole to about 10,000 grams per mole, about 2,500 grams per mole to about 8,000 grams per mole, or about 3,000 grams per mole to about 6,000 grams per mole. In other embodiments, the second PEG may have an average molecular weight of about 2,500 grams per mole to about 20,000 grams per mole, about 3,000 grams per mole to about 15,000 grams per mole, or about 3,500 grams per mole to about 10,000 grams per mole.

In some embodiments, two polymeric solvents may be utilized in a defined ratio. For example, when a first, low molecular weight PEG and a second, high molecular weight PEG is used, the first PEG and the second PEG may be combined in a ratio of 4:1 to 1:2, 3:1 to 1:1, or 2.5:1 to 1.5:1.

The one or more polymeric solvents preferably are present in a total amount of about 0.1% to about 30% by weight, based on the total weight of the oral composition. The total amount may be accounted for by a single polymeric solvent or may be the combination of two or more polymeric solvents. Preferably, the polymeric solvent(s) are present in a total amount of about 1% to about 25% by weight, about 5% to about 20% by weight, or about 8% to about 15% by weight, based on the total weight of the oral composition. In some embodiments, two polymeric solvents can be used, each of which is independently present in an amount of about 2% to about 15% or about 2% to about 10% by weight based on the total weight of the oral composition. For example, the oral composition can comprise a first polyalkylene glycol, such as PEG, having a low molecular weight (as defined herein) in an amount of about 2% to about 15% or about 5% to about 12% by weight and also comprise a second polyalkylene glycol, such as PEG, having a high molecular weight (as defined herein) in an amount of about 2% to about 10% or about 3% to about 8% by weight, based on the total weight of the oral composition.

In one or more embodiments, an aqueous medium may also be utilized in forming the oral composition. The aqueous medium preferably comprises water, such as substantially pure water; however, salts, buffers, or the like also may be included in the aqueous medium. In some embodiments, the aqueous medium can comprise about 0.5% to about 15% by weight, about 1% to about 14%, about 2% to about 13%, about 5% to about 12%, or about 7% to about 11% by weight of the overall oral composition. The water content of the oral composition may be particularly relevant to the composition in its final form. For example, when the oral composition is provided in a film form, it can be particularly useful for the film to be dried to a final moisture content or final water content, and such moisture content or water content can be within a range as defined above. The moisture level affects the properties of the film and its manufacture. When the moisture content of the composition is above 15% by weight, the film may have insufficient tensile strength and be too pliable. When the moisture content is below 0.5% by weight, the film may be too brittle.

The oral composition, in some embodiments, can include one or more opacifying agents. Suitable opacifiers can include any material suitable for causing a solid form of the oral composition to be substantially opaque. For example, metal oxides may be utilized as opacifiers, and specific, non-limiting examples of suitable metal oxides include titanium dioxide, zinc oxide, and iron oxide. Examples of other materials suitable for use as an opacifier include magnesium carbonate, talc, and the like. One or more opacifiers, and particularly one or more metal oxides, may be included in the oral composition in a total amount of about 0.02% to about 2% by weight, about 0.05% to about 1.5% by weight, or about 0.08% to about 1% by weight, based on the total weight of the composition.

In various embodiments, the oral composition can further comprise one or more additional components. The one or more optional components may be selected from one or more of the group comprising, a pH adjusting agent, a flavouring agent, a sweetener, a plasticizer, a surfactant, a preservative, a colouring agent and a delivery enhancing polymer.

The pH adjusting agent may be selected from the group comprising, sodium hydroxide, potassium hydroxide, citric acid, lactic acid, phosphoric acid and sodium bicarbonate. Preferably the pH adjusting agent is sodium hydroxide. Too low a pH may result in the undesirable etching of teeth. The oral composition may have a pH in the range of from 5 to 6.5.

The plasticizer is a physiologically acceptable plasticizer. In one embodiment, the plasticizer is a hydrophilic plasticizer, such as one or more of the group comprising a polyol such as glycerol, and monosaccharides, oligosaccharides, lipids, sorbitol and sorbitan. The preferred plasticizer is glycerol. Preferably the plasticizer is present in a proportion of from 0.25% to 15% by weight of the oral composition. Preferably, the plasticizer(s) are present in a total amount of about 0.5% to about 8% by weight, about 0.75% to about 6% by weight, or about 1% to about 5% by weight, based on the total weight of the oral care composition. In some embodiments, two plasticizers can be used, each of which is independently present in an amount of about 0.5% to about 5% or about 1% to about 4% by weight based on the total weight of the oral care composition. For example, the oral care composition can comprise glycerol (or another polyol) in an amount of about 0.5% to about 5% or about 1% to about 4% by weight, based on the total weight of the oral care composition

The surfactant may be an ionic surfactant or a non-ionic surfactant. Preferably the non-ionic surfactant comprises one or more fatty acids, which may be saturated or unsaturated. More preferably the non-ionic surfactant comprises at least one unsaturated fatty acid such as oleic acid and/or linoleic acid and optionally at least one saturated fatty acid such as palmitic acid and/or stearic acid. Preferably, if present, the surfactant is in a proportion of from 0.01 to 5 wt. % by dry weight of the oral composition.

A polymeric compound which enhances the delivery of the active agent may also be present. Examples of such delivery enhancing polymers include copolymers of polyvinylmethylether with maleic anhydride.

The oral composition can comprise at least one of a sweetener and a flavoring agent. In some embodiments, a sweetener can be present in an amount in the range of about 0.1% to about 1% by weight, or about 0.2% to about 0.5% by weight, based on the total weight of the oral composition. The sweetener may be a sugar substitute, such as an artificial sugar substitute or a natural sugar substitute. The artificial sugar substitute may be selected from the group comprising sucralose, aspartame, advantame, saccharin, acesulfame potassium and cyclamate, with sucralose being preferred. The natural sugar substitute may be selected from the group comprising erythritol, mannitol, stevia, sorbitol and xylitol. Preferably the sweetener is sucralose.

In some embodiments, a flavoring agent can be present in an amount of about 0.1% to about 2% by weight, or about 0.5% to about 1% by weight, based on the total weight of the oral composition. The flavouring agent may be an artificial flavouring or a natural flavouring. The flavouring agent may be one or more selected from the group comprising herb flavouring, such as mint, for instance spearmint or peppermint, a spice flavouring such as ginger, cinnamon or vanilla, or a fruit flavouring such as apple, grape, orange, pear or strawberry. Preferably the flavouring agent is peppermint.

In further embodiments, the oral composition can comprise one or more preservatives. Non-limiting examples of suitable preservatives include butylated hydroxytoluene (BHT), butylated hydroxyanisole (BHA), potassium sorbate, sodium sorbate, sodium benzoate, and the like. One or more preservatives can be included in the oral composition in a total amount of about 0.02% to about 2% by weight, about 0.05% to about 1.5% by weight, or about 0.08% to about 1% by weight, based on the total weight of the composition.

Still further, the oral composition can include one or more natural or artificial coloring agents as desired, preferably in an amount of 0.001% by weight to about 0.2% by weight, or about 0.01% by weight to about 0.1% by weight. The colouring agent may be FD & C blue no. 1 and lakes thereof.

The present oral compositions can be particularly beneficial to deliver active agents to areas of the oral or buccal cavity. In such embodiments, the present oral compositions, particularly when on a surface of the oral cavity, are effective to deliver the active agent for an extended duration of time. While it is desirable for the oral composition to be dissolvable within the oral or buccal cavity, the oral composition must persist in the oral or buccal cavity for a sufficient time to provide a useful effect. Preferably, the present oral composition dissolves within the oral or buccal cavity after application to a surface thereof and provides a desired effect (the effect consistent with the mode of action of the active agent) for a time of at least 15 minutes, at least 30 minutes, or at least 45 minutes (e.g., up to a time of about 4 hours, up to a time of about 3 hours, or up to a time of about 2 hours). In some embodiments, the present oral composition dissolves within the mouth and provides an effect consistent with the mode of action of the active agent for a time of about 15 minutes to about 180 minutes, about 15 minutes to about 150 minutes, or about 15 to about 120 minutes or about 45 minutes to about 120 minutes.

The ability to provide the desired effect from delivery of the active agent can depend upon the ability of the oral composition to persist within the oral or buccal cavity, particularly being substantially adhered to a specific area of a surface of the oral or buccal cavity, for a sufficiently long period of time. It is generally understood that the oral composition, when adhered to a surface of the oral or buccal cavity in a substantially solid form, will be expected to dissolve over time due to contact with saliva and/or other mucosal secretions within the oral or buccal cavity. Preferably, a solid form of the oral composition when substantially adhered to a surface of the oral or buccal cavity can remain in a semi-solid or solid state for a time of about 5 minutes or greater, about 20 minutes or greater, about 30 minutes or greater, or about 45 minutes or greater. It is understood that the solid form of the oral composition will be expected to substantially completely dissolve within the oral or buccal cavity within a maximum time, such as a maximum time of about 4 hours, about 3 hours, or about 2 hours. In certain embodiments, the oral composition can remain in a semi-solid or solid state when in the oral or buccal cavity for a time of about 15 minutes to about 180 minutes, about 15 minutes to about 120 minutes, about 20 minutes to about 100 minutes, or about 30 minutes to about 90 minutes. As such, the oral composition, when present in the oral or buccal cavity in a substantially solid form, such as a film, can be configured to dissolve substantially completely when in contact with mucosa in a time of about 15 minutes to about 120 minutes.

In one or more embodiments, the present disclosure further provides methods of making oral compositions as described herein. Preferably, the methods comprise first formulating a premix that includes the one or more active agents and the one or more polymeric solvents. Specifically, this can include solubilizing the one or more active agents in the one or more polymeric solvents while heating or while heating and mixing. This can be achieved, for example, using a hotplate and a homogenizer. The combination of the active agent(s) and the polymeric solvent(s) can be heated, for example, to a temperature of about 50° C. to about 120° C., about 60° C. to about 115° C., or about 70° C. to about 110° C. to form the premix. It is understood that not all active agents will necessitate formation of a premix. Moreover, one or more active agents may be included in a premix, and one or more active agents may be added to the main mixture.

After the premix has been formed (i.e., the active agent(s) have been substantially completely dissolved in the polymeric solvent(s) used), the remaining components of the oral composition can be mixed into the premix to form the main mixture. At a minimum, the method of preparation can comprise mixing into the premix at least one or more film forming polymers, one or more bioadhesive agents, and water (or another aqueous solvent) to form a liquid composition. The liquid composition will be a viscous liquid, and mixing thus may be carried out using, for example, a high shear vacuum mixer to achieve mixing and degassing of the solution to form a liquid composition. Mixing is preferably carried out until a substantially homogeneous mixture is achieved and all of the components of the oral composition have been incorporated into the solution.

The so-formed liquid composition can then be processed into the desired end form of the oral composition. For example, the composition may be placed into molds to form pastilles or poured into cooling pans to form sheets that may be cut to desired sizes. In certain embodiments, to facilitate forming of films, the liquid composition can be coated onto a backing sheet to form a layer of the composition having the desired thickness as otherwise described herein. The backing sheet preferably is coated with a release layer, such as a wax or similar coating that will all of the formed film to be readily released therefrom. A knife or other blade may be used to coat the liquid composition to the desired thickness. Preferably, the film has a sufficient viscosity such that, after coating onto the backing sheet, the liquid composition will not substantially spread and will thus remain substantially consistent in thickness across the total surface of the coated liquid composition.

The liquid composition finally can be dried to the desired total water content as otherwise described herein to achieve the end product in a substantially solid form. Drying can comprise applying heated air to the layer of the composition on the backing sheet. Such heated air may be provided at a temperature that is above ambient and up to a temperature that is below the melting point of the film forming polymer(s). For example, the air can be a temperature of about 30° C. to about 60° C. If desired, ambient temperature air or even cooled air may be applied to facilitate drying of the film. The drying air may be applied for a time of about 5 minutes to about 150 minutes, typically about 20 minutes to about 120 minutes. The oral composition can be configured such that drying takes a minimum amount of time regardless of the application of drying air. Thus, while the application of the drying air can expedite drying, the film will typically take a time of at least 15 minutes, at least 20 minutes, at least 30 minutes, or at least 40 minutes to achieve the desired water content and thus be a self-supporting, solid composition that is no longer tacky and may be further processed for packaging of the product. In particular, drying may require a time of about 15 minutes to about 240 minutes, about 30 minutes to about 220 minutes, about 45 minutes to about 200 minutes, or about 60 minutes to about 150 minutes. In some embodiments, the drying air may be applied from all sides, such as both above and below the layer of the composition or only from below the layer of the composition or only from above the layer of the composition (and, as such, application of air from below the layer of the composition (i.e., application of air to the backing layer) may be excluded). As a non-limiting example, drying of the film may be carried out by passing the layer of the composition on the backing sheet through a drying tunnel. In such tunnel, drying air may be applied along the entire length of the drying tunnel or only at a portion of the tunnel. Drying air may be forced air (e.g., provided via fans or blowers). In some embodiments, convection heating may be used for drying. As such, heating elements above the film layer may generate convective currents that facilitate drying of the film.

After the film has dried, the film may be cut to desired dimensions. For example, this may comprise cutting the film of the oral composition into individual strips having a width of about 10 mm to about 25 mm and having a length of about 10 mm to about 85 mm. Cutting to other dimensions as otherwise described herein is also encompassed. The individual film strips may be packaged as desired into containers with a defined number of strips per package.

The oral composition or film obtainable by the method of making disclosed herein may be used in a method of administering an active agent to an oral or buccal cavity. The method comprises at least the step of:
applying the oral composition obtained as described herein to a portion of an oral or buccal cavity.
   When the active agent is a bleaching agent, the method may be a cosmetic method, preferably a solely cosmetic method.

The oral composition may first be released from its sterile packaging. Subsequently, any backing sheet is removed, for instance by peeling the oral composition or film from the backing sheet. Once the oral composition is free from any other layers, it may be applied to a portion of an oral or buccal cavity.

Embodiments of the presently disclosed oral composition were subjected to consumer acceptability testing to evaluate effectiveness and desirability of the product. The present oral composition is comfortable to use in the oral cavity. In addition, the oral composition films provide a soft, comfortable barrier. This created an advantageous difference over known, topical treatments. Whereas known topical treatments for oral use have been found to produce effects consistent with their mode of action over substantially the entire mouth, tongue, and throat due to their dislocation from the point of application and/or excessive dripping, the present oral compositions overcome such problem by remaining substantially in place once adhered to a localized site within the oral cavity. As such, only localized treatment is provided to the actual area of application with minimal effect to other areas of the oral cavity. Solid forms of the present oral composition, such as films in particular, can be easily molded and applied over the area of the oral cavity where treatment is needed. The film easily adheres to the tooth, gum, or other oral mucosal surface and thereby provides delivery of the active agent to the desired site. To this end, the film is preferably configured to be repositionable for a minimum period of time to allow for ease of application. For example, the film can be repositionable for a time of up to 2 minutes, such as about 5 seconds to about 2 minutes, about 10 seconds to about 1 minute, or about 15 seconds to about 45 seconds. Repositionable means that the film can be removed and reapplied or can be slid along the oral surface without substantial tearing or other degradation of the film. Once the film has adhered to the oral surface and is no longer considered repositionable, the film is substantially unable to be moved or removed and reapplied without tearing or otherwise destroying the film. Preferably, once the oral composition film has been applied and adhered to the surface of the oral cavity, the film remains in place for the duration of effectiveness of the product (i.e., until complete dissolution of the product), and the film is not dislodged from its location by activities such as talking or drinking.

### EXAMPLES

Table 1 below provides ingredients included in an embodiment of the oral composition of the present invention. The ingredients are listed by order of addition. Table 1 also includes the weight percentage of each ingredient, based on the total weight of the oral composition. The primary function of each ingredient is also included.

TABLE 1
Oral Composition Formula 1

| | | | Weight |
|---|---|---|---|
| Batch | Ingredient | Function | Percentage |
| Premix | PEG 400 | Polymeric solvent | 2-15 wt. % |
| Premix | PEG 4000 | Polymeric solvent | 2-10 wt. % |
| Premix | Active agent | Active agent | 0.01-35 wt. % |
| Main | Polyvinylpyrrolidone | Film forming polymer | 20-40 wt. % |
| Main | Pullulan | Film forming polymer | 20-40 wt. % |
| Main | Polycarbophil | Bioadhesive agent | 0.75-9 wt. % |
| Main | Glycerol | Plasticizer | 0.75-9 wt. % |
| Main | Potassium sorbate | Preservative | 0.2-2 wt. % |
| Main | Titanium dioxide | Opacifier | 0.2-2 wt. % |
| Main | Brilliant Blue | Colorant | 0.001-0.02 wt. % |
| Main | Water | Solvent | 0.05-15 wt. % |

### Examples 1 to 11

Films comprising the oral composition of the present invention containing 11 different active agents were prepared from a single master batch of casting liquid and are presented as Examples 1 to 11.

The master batch of casting liquid was prepared as follows according to the proportions shown in Table 2 below. Water and glycerol were weighed into a 5 L stainless steel pot. PEG 400 was weighed into a 250 ml stainless steel pot. All other components were weighed into weighing boats. Pullulan was added slowly over 20 minutes to the water/glycerol at speed setting 3 on an IKA Ultra Turrax T50 homogeniser. Kollidon was then added over 20 minutes mixing at setting 4 followed by the potassium sorbate and titanium dioxide which were added and mixed for 5 minutes at setting 4. The Noveon AA-1 was then added and mixed at setting 5 over 15 minutes. Finally, the PEG400 and PEG 4000 were added and mixed over 8 minutes at setting 5 of the Ultra Turrax to produce a uniform casting liquid. The casting liquid was degassed under vacuum and then stored at ambient temperature for further processing.

TABLE 2
Master batch casting liquid composition

| | | Actual Batch | % w/w |
|---|---|---|---|
| Ingredient | Supplier | Weight (g) | Dry |
| Kollidon 90 F (PVP) | BTC Europe | 265.62 | 37.78 |
| Pullulan | Nagase | 265.49 | 37.78 |
| Noveon AA-1 | Surfachem | 15.98 | 2.27 |
| Glycerol | Oleon | 19.73 | 2.81 |
| Potassium Sorbate | Azelis | 1.07 | 0.15 |
| Titanium Dioxide | Azelis | 1.14 | 0.16 |
| PEG 400 | Sigma | 86.65 | 12.32 |
| PEG 4000 | Sigma | 47.39 | 6.72 |
| Purified Water | VWR | 2334.51 | — |
| Total | | 3037.58 | 100 |

The master batch with the composition shown in Table 2 was then split into 12 separate 200 g portions and to each portion a known amount of an active ingredient was added as described below. Table 3 details the type and proportion of the various active ingredients added to the formulation.

The designated quantity of active agent was added to 200 g of the master batch of casting liquid and mixed either by hand or using an IKA Ultra Turrax T50 at speed setting 3-3.5, then degassed under vacuum until a smooth, uniform casting liquid was achieved. From each casting liquid a sheet of film was cast at thickness of 0.75 mm using a Sheen 1133N Film Coater with stainless steel knife onto PE coated paper. Each film was dried under an air drying hood for 25-40 mins.

Each of the casting liquids produced were deemed to be of a good quality and each film produced was visually assessed and deemed to be of a high quality, i.e. an evenly dried, single-phase film with sufficient tensile strength to be further processed.

TABLE 3
Active agent and proportion added to master
batch portion

| | | | | Amount added |
|---|---|---|---|---|
| | | | | to 200 g of |
| Example | Active agent | Supplier | Function | master batch |
| 1 | Peroxydone | ISP | Bleaching Agent | 27.0 g |
| | K90 | | | |
| 2 | Melatonin | Medex | Natural hormone | 0.8 g |
| 3 | Vitashure | Balchem | Encapsulated | 23.1 g |
| | 301 | | caffeine | |
| 4 | Optaflow | Azelis | Salivating agent | 5.2 g |
| 5 | Ivy Leaf | Finzelberg | Herbal Remedy | 5.5 g |
| | Extract | | | |
| 6 | Potassium | Flukka | Bleaching Agent | 2.5 g |
| | oxalate | | | |
| 7 | Vitryxx | Schott | Bioactive glass | 2.5 g |
| 8 | Eureco | Harke | Bleaching Agent | 27.0 g |
| 9 | Clove Oil | Sigma | Essential Oil | 2.5 g |
| 10 | Simethicone | S Black | Anti-foaming | 2.5 g |
| | | | agent | |
| 11 | Spherulites | Soliance | Anti-snoring | 2.5 g |
| | | | agent | |

### Example 12

Films comprising the oral composition of the present invention and containing the active ingredient cannabidiol were manufactured comprising the formulation detailed in Table 4 below.

TABLE 4
Cannabidiol film composition

| Ingredient | Supplier | Batch weight (g) | % w/w dry |
|---|---|---|---|
| Kollidon 90 F (PVP) | BTC Europe | 20.41 | 34.01 |
| Pullulan | Nagase | 20.41 | 34.01 |
| Noveon AA-1 | Lubrizol | 1.23 | 2.05 |
| Glycerol | Brenntag | 1.52 | 2.53 |
| Potassium Sorbate | Azelis | 0.08 | 0.13 |
| Titanium Dioxide | Merck | 0.09 | 0.15 |
| Brilliant Blue | Claremont | 0.01 | 0.01 |
| PEG 400 | Merck | 6.66 | 11.09 |
| PEG 4000 | Sigma | 3.63 | 6.06 |
| Cannabidiol resin | CBD oil Europe | 5.98 | 9.96 |
| Water | VWR | 139.99 | — |
| Total | | 200.01 | 100.00 |

All liquid materials (water, PEG 400, cannabidiol resin, glycerol, brilliant blue) were weighed and added to a stainless steel container. Pullulan was added and mixed using an IKA Ultra Turrax T50 at speed setting 3 for 3 minutes. Kollidon 90 F was added to the pot and mixed at speed setting 4 for 6 minutes with the Noveon AA-1 then added and mixed at speed setting 4.5 for 4 minutes. The remaining ingredients of titanium dioxide, PEG 4000 and potassium sorbate were added and mixed at speed setting 5 for 5 minutes. The casting liquid was held overnight at ambient conditions to degas the mixture and a uniform mixture was obtained with no separation observed.

From the casting liquid a sheet of film was cast at thickness of 0.6 mm using a Sheen 1133N Film Coater with stainless steel knife onto PE coated paper. The film was dried under an air drying hood for 20 minutes and then cut into rectangular strips of dimensions 22 mm by 32 mm and mass of 120 mg which contained a theoretical quantity of cannabidiol of approximately 10 mg.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. An oral composition comprising:
one or more film forming polymers in a total amount of about 40% to about 80% by weight;
one or more bioadhesive agents in a total amount of about 0.5% to about 10% by weight;
one or more active agents in a total amount of about 0.01% to about 35% by weight;
one or more polymeric solvents in which the one or more active agents is solubilized, the one or more polymeric solvents being in a total amount of about 1% to about 30% by weight; and
an aqueous medium in an amount of about 0.5% to about 15% by weight,
each of the foregoing amounts being based on the total weight of the oral composition,
wherein the oral composition is in the form of a film having a thickness of about 50 μm to about 500 μm and wherein the oral composition does not comprise benzocaine.

2. The oral composition of claim 1, wherein the one or more film forming polymers comprises one or more of a polyvinylpyrrolidone and a polysaccharide.

3. The oral composition of claim 1, wherein the one or more film forming polymers is selected from the group consisting of a polyvinylpyrrolidone, pullulan, pectin, starch, carboxyalkyl cellulose or a salt thereof, hydroxyalkyl cellulose or a salt thereof, in which the alkyl group is independently selected from C₁₋₅ alkyl, alginic acid, salt of alginic acid, pectin, and combinations thereof.

4. The oral composition of claim 1, wherein the one or more film forming polymers includes a polyvinylpyrrolidone in an amount of about 20% to about 40% by weight and pullulan in an amount of about 20% to about 40% by weight, based on the total weight of the oral composition.

5. The oral composition of claim 1, wherein the one or more bioadhesive agents comprises one or more of a polyacrylic acid and derivatives thereof and a gum.

6. The oral composition of claim 1, wherein the one or more bioadhesive agents includes a polycarbophil in an amount of about 0.25% to about 5% by weight and the composition further comprises glycerol in an amount of about 0.25% to about 5% by weight, based on the total weight of the oral composition.

7. The oral composition of claim 1, wherein the one or more active agents is one or more selected from the group comprising a pharmaceutical, a bleaching agent, an anti-staining agent, a dental remineralization agent, a dental desensitizing agent, an anti-caries agent, an anti-malodour agent, herbal remedy, essential oil, a botanical extract of cannabinoids or an anti-calculus agent, with the proviso that the active agent does not comprise benzocaine.

8. The oral composition of claim 1, wherein the one or more active agents are present in an amount of about 2% to about 15% by weight.

9. The oral composition of claim 1, wherein the one or more polymeric solvents comprises a lower molecular weight polyalkylene glycol and a higher molecular weight polyalkylene glycol in which the alkylene group of each polyalkylene glycol is independently selected from C₂-C₅ alkylene.

10. The oral composition of claim 1, wherein the one or more polymeric solvents includes a first polyethylene glycol (PEG) having a molecular weight of 1,500 grams per mole or less and a second polyethylene glycol (PEG) having a molecular weight of 2,000 grams per mole or greater.

11. The oral composition of claim 10, wherein the first PEG is present in an amount of about 1% to about 20% by weight, and the second PEG is present in an amount of about 1% to about 20% by weight, based on the total weight of the oral composition.

12. The oral composition of claim 1, wherein the aqueous medium is present in an amount of about 5% to about 12% by weight, based on the total weight of the oral composition.

13. The oral composition of claim 1, wherein the composition further comprises one or more opacifiers in a total amount of about 0.02% to about 2% by weight based on the total weight of the composition.

14. The oral composition of claim 13, wherein the opacifier includes titanium dioxide.

15. The oral composition of claim 1, wherein the film is configured to dissolve substantially completely in contact with oral or buccal mucosa in a time of about 15 minutes to about 120 minutes.

16. A method of making an oral composition comprising:
solubilizing one or more active agents in one or more polymeric solvents while heating to a temperature of about 50° C. to about 120° C. to form a premix;
mixing into the premix each of the following components to form a liquid composition:
one or more film forming polymers;
one or more bioadhesive agents; and
water;
coating the liquid composition onto a backing sheet to form a layer of the composition having a thickness of about 100 μm to about 3,000 μm; and
drying the layer to form a film of the oral composition having a total water content of about 5% to about 15% by weight based on the total weight of the film.

17. The method of claim 16, wherein the film of the oral composition comprises:
a total of about 0.01% to about 35% by weight of the one or more active agents;
a total of about 1% to about 30% by weight of the one or more polymeric solvents:
a total of about 40% to about 80% by weight of the one or more film forming polymers; and
a total of about 0.5% to about 10% by weight of the one or more bioadhesive agents,
each of the foregoing amounts being based on the total weight of the film of the oral composition.

18. The method of claim 16, wherein the drying comprises applying heated air to the layer of the composition on the backing sheet for a time of about 20 minutes to about 120 minutes.

19. The method of claim 18, wherein the heated air is applied from one or both of from above and from below the layer of the composition.

20. The method of claim 18, wherein the drying is carried out by passing the layer of the composition on the backing sheet through a drying tunnel.

21. The method of claim 16, further comprising cutting the film of the oral composition into individual strips having a width of about 10 mm to about 20 mm and having a length of about 10 mm to about 85 mm.
